(19) United States
(12) Patent Application Publication
(10) Pub. No.: US 2024/0009146 A1
(43) Pub. Date: Jan. 11, 2024

(54) CYCLOBENZAPRINE TREATMENT FOR ALCOHOL USE DISORDER

(71) Applicant: Tonix Pharmaceuticals Holding Corp., Chatham, NJ (US)

(72) Inventors: Seth Lederman, South Dartmouth, MA (US); Greg M. Sullivan, Barryville, NY (US)

(21) Appl. No.: 18/037,815

(22) PCT Filed: Nov. 19, 2021
(86) PCT No.: PCT/US2021/060011

§ 371 Date: May 19, 2023

Related U.S. Application Data
(60) Provisional application No. 63/116,777, filed on Nov. 20, 2020.

(51) Int. Cl. A61K 31/137

(52) U.S. Cl. CPC A61K 31/137

(57) ABSTRACT

The present disclosure provides a method for treating alcohol use disorder (AUD) and associated symptoms to a subject in need thereof by administering to the subject a pharmaceutical composition comprising a therapeutically effective amount of cyclobenzaprine or a pharmaceutically acceptable salt thereof and a pharmaceutically acceptable carrier. The pharmaceutically acceptable salt comprises a cyclobenzaprine acid salt.

### BACKGROUND OF THE INVENTION

Cyclobenzaprine, or 3-(5H-dibenzola[a,d]cyclohepten-5-ylidene)-N,N-dimethyl-1 propanamine, was first approved by the U.S. Food and Drug Administration in 1977 for the treatment of acute muscle spasms of local origin. (Katz and Dube, 1988).

The utility of a very low dose cyclobenzaprine as an agent for improving the quality of sleep, as a sleep deepener, or for treating sleep disturbances has previously been investigated. See, e.g., U.S. Pat. App No. US20110124656A1, and U.S. Pat. Nos. 6,395,788 and 6,358,944, incorporated herein by reference.

International Publication No. WO2013/188847, incorporated herein by reference, discloses a low dose, sublingual formulation of cyclobenzaprine (TNX-102 SL) that provides rapid transmucosal absorption into the blood and uniquely reduces production of a long half-life active metabolite, norcyclobenzaprine, due to its bypass of first-pass hepatic metabolism.

TNX-102 SL comprises cyclobenzaprine and a basifying agent. See, e.g., WO2013/188847, incorporated herein by reference.

An estimated 36 million adults in the United States have alcohol use disorder (AUD), a chronic relapsing brain disease characterized by compulsive alcohol use, loss of control over alcohol intake, and a negative emotional state when not using alcohol. There is an unmet need to treat alcohol use disorder and the symptoms associated with AUD. As described in the disclosure, cyclobenzaprine (e.g., TNX-102 SL) meets this unmet need and improves sleep quality and the rate of successful recovery for subjects suffering from AUD, as well as before, during or after detoxification (detox).

### SUMMARY OF THE INVENTION

The present disclosure provides a method for treating alcohol use disorder and associated symptoms to a subject in need thereof.

A first aspect of the disclosure provides a method for treating alcohol use disorder (AUD) and associated symptoms, comprising administering to a subject in need thereof, a pharmaceutical composition comprising a therapeutically effective amount of cyclobenzaprine or a pharmaceutically acceptable salt thereof and a pharmaceutically acceptable carrier.

In some embodiments, the pharmaceutical composition used in the method of the disclosure is administered one or more times daily.

In some embodiments, the pharmaceutical composition used in the method of the disclosure comprises between 0.1 mg and 30 mg of cyclobenzaprine or a pharmaceutically acceptable salt thereof. In some embodiments, the pharmaceutical composition used in the method of the disclosure comprises between 1 mg and 20 mg of cyclobenzaprine or a pharmaceutically acceptable salt thereof. In some embodiments, the pharmaceutical composition used in the method of the disclosure comprises less than 10 mg of cyclobenzaprine or a pharmaceutically acceptable salt thereof. In some embodiments, the pharmaceutical composition used in the method of the disclosure comprises less than 5 mg of cyclobenzaprine or a pharmaceutically acceptable salt thereof. In some embodiments, the pharmaceutical composition used in the method of the disclosure comprises about 5.6 mg of cyclobenzaprine or a pharmaceutically acceptable salt thereof. In some embodiments, the pharmaceutical composition used in the method of the disclosure comprises about 2.8 mg of cyclobenzaprine or a pharmaceutically acceptable salt thereof. In some embodiments, the pharmaceutical composition used in the method of the disclosure comprises about 5.6 mg of a cyclobenzaprine salt. In some embodiments, the pharmaceutical composition used in the method of the disclosure comprises about 2.8 mg of a cyclobenzaprine salt. In some embodiments, the pharmaceutical composition used in the method of the disclosure comprises about 5.6 mg of a cyclobenzaprine acid salt. In some embodiments, the pharmaceutical composition used in the method of the disclosure comprises about 2.8 mg of a cyclobenzaprine acid salt. In some embodiments, the pharmaceutical composition used in the method of the disclosure comprises about 5.6 mg of cyclobenzaprine HCl. In some embodiments, the pharmaceutical composition used in the method of the disclosure comprises about 2.8 mg of cyclobenzaprine HCl.

In some embodiments, the pharmaceutical composition used in the method of the disclosure is administered as two dosage units, and wherein each dosage unit comprises 2.8 mg of cyclobenzaprine or a pharmaceutically acceptable salt thereof. In some embodiments, the pharmaceutical composition used in the method of the disclosure is administered simultaneously as two dosage units, and wherein the combined amount in the two dosage units is 5.6 mg of cyclobenzaprine or a pharmaceutically acceptable salt thereof. In some embodiments, the pharmaceutical composition used in the method of the disclosure is administered once daily.

In some embodiments, the pharmaceutical composition used in the method of the disclosure is administered sublingually, buccally, orally, intravenously, intramuscularly, subcutaneously, inhalationally, intranasally, transdermally, parenterally, rectally, or vaginally. In some embodiments, the pharmaceutical composition used in the method of the disclosure is administered by transmucosal absorption. In some embodiments, the pharmaceutical composition used in the method of the disclosure is administered sublingually.

In some embodiments, the pharmaceutical composition used in the method of the disclosure comprises a cyclobenzaprine acid salt and further comprises a basifying agent. In some embodiments, the cyclobenzaprine acid salt is cyclobenzaprine HCl.

In some embodiments, the cyclobenzaprine in the pharmaceutical composition used in the method of the disclosure comprises at least in part a eutectic of cyclobenzaprine and mannitol.

In some embodiments, the pharmaceutical composition used in the method of the disclosure is administered in combination with behavioral or environmental intervention.

In some embodiments, the pharmaceutical composition used in the method of the disclosure is administered before, during, or after detoxification.

### DETAILED DESCRIPTION OF INVENTION

Unless otherwise defined herein, scientific and technical terms used in this application shall have the meanings that are commonly understood by those of ordinary skill in the art. In case of conflict, the present specification, including definitions, will control.

Throughout this specification and embodiments, the word “comprise,” or variations such as “comprises” or “comprising,” will be understood to imply the inclusion of a stated integer or group of integers but not the exclusion of any other integer or group of integers.

The term “including” or “includes” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members.

Exemplary methods and materials are described herein, although methods and materials similar or equivalent to those described herein can also be used in the practice or testing of the various aspects and embodiments. The materials, methods, and examples are illustrative only and not intended to be limiting.

### Definitions

In order that the disclosure may be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

In one aspect, the application discloses a method for treating alcohol use disorder and associated symptoms thereof. In other aspects, the method is used before, during or after detoxification. The symptom may be, but is not limited to, a sleep disturbance or a non-sleep disturbance.

The method comprises administering to a subject in need or at risk thereof, a pharmaceutical composition comprising a therapeutically effective amount of cyclobenzaprine and a pharmaceutically acceptable carrier. The method comprises administering to a subject in need or at risk thereof, a pharmaceutical composition comprising a therapeutically effective amount of a pharmaceutically acceptable salt of cyclobenzaprine and a pharmaceutically acceptable carrier.

As used herein, the term “treat” and its cognates refer to a full or partial amelioration or modulation of alcohol use disorder or at least one discernible symptom thereof in alcohol use disorder. In some embodiments, “treat” refers to an improvement (i.e., reduction) in the amount of alcohol consumed. In some embodiments, “treat” refers to wanting to cut down on how much alcohol is consumed or making successful attempts to do so. In some embodiments, “treat” refers to spending less time drinking, getting alcohol, or recovering from alcohol use. In some embodiments, “treat” refers to feeling a reduced craving or urge to drink alcohol. In some embodiments, “treat” refers to being able to better fulfill major obligations at work, school or home. In some embodiments, “treat” refers to improvement from continuously drinking alcohol although it is causing physical, social or interpersonal problems. In some embodiments, “treat” refers to improvement, i.e., less, giving up or reducing social and work activities and hobbies. In some embodiments, “treat” refers to improvement, i.e., less, use of alcohol in situations where it is not safe. In some embodiments, “treat” refers to improvement, i.e., less, developed tolerance to alcohol. In some embodiments, “treat” refers to improvement in withdrawal symptoms (e.g. nausea, sweating, shaking, and problem sleeping).

As used herein, the term “cyclobenzaprine” refers to cyclobenzaprine or a metabolite thereof, prodrugs of cyclobenzaprine or a metabolite thereof. Metabolites of cyclobenzaprine useful according to the methods of this application are metabolites that have substantially the same or better activity than cyclobenzaprine in alleviating alcohol use disorder or associated symptoms thereof. Cyclobenzaprine metabolites that may be useful according to this application include CBP 10,11-trans-dihydriol, N-desmethyl-2-hydroxycyclobenzaprine, 3-hydroxycyclobenzaprine, N-desmethylcyclobenzaprine, cyclobenzaprine N-oxide, or a chiral isomer of these metabolites. A prodrug of cyclobenzaprine is a derivative of cyclobenzaprine that is metabolized in vivo into the active agent. Prodrugs useful according to this application are those that have substantially the same or better activity than cyclobenzaprine in treating alcohol use disorder and associated symptoms thereof. Methods for making prodrugs are readily known in the art (e.g., Balant, et al. 1990 and Bund-gaard, H et al. 1991 incorporated by reference herein).

In some embodiments of this disclosure, the cyclobenzaprine is combined with a basifying agent. In other embodiments, the cyclobenzaprine in that combination is an acid salt of cyclobenzaprine. In other embodiments, the acid salt of cyclobenzaprine is cyclobenzaprine HCl. See, e.g., WO2013/188847, incorporated herein by reference.

The “basifying agent” included in some embodiments of this disclosure is selected from a group consisting of potassium dihydrogen phosphate (monopotassium phosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), bicarbonate or carbonate salts, dipotassium citrate, tripotassium citrate, disodium citrate, trisodium citrate, borate, hydroxide, silicate, nitrate, dissolved ammonia, the conjugate bases of some organic acids (including bicarbonate), and sulfide. In some embodiments, the basifying agent is potassium dihydrogen phosphate (monopotassium phosphate, monobasic potassium phosphate, KH₂PO₄) or dipotassium hydrogen phosphate (dipotassium phosphate, dibasic potassium phosphate, K₂HPO₄). In some embodiments, the basifying agent is sometimes an ingredient (and excipient) in a tablet, and the basifying agent exerts its effects during the time the tablet is being dispersed in the mucous material, while parts of the formulation are dissolving in the mucous material and for a period of time after the tablet is dissolved in the mucous material. Surprisingly, the addition of a basifying agent to a composition of the invention improves the pharmacokinetic properties of the composition. This is exemplified by cyclobenzaprine HCl as one embodiment of a cyclobenzaprine useful in the methods and compositions of this disclosure. A basifying agent with particular effects on cyclobenzaprine HCl is dipotassium hydrogen phosphate (K₂HPO₄). Another basifying agent with particular effects on cyclobenzaprine HCl is potassium dihydrogen phosphate (KH₂PO₄). Another basifying agent with particular effects on cyclobenzaprine HCl is disodium hydrogen phosphate (Na₂HPO₄). Another basifying agent with particular effects on cyclobenzaprine HCl is tripotassium citrate. Another basifying agent with particular effects on cyclobenzaprine HCl is trisodium citrate.

In some embodiments of this disclosure, the cyclobenzaprine or cyclobenzaprine acid salt, e.g., cyclobenzaprine HCl, is at least in part contained in a eutectic system with mannitol. See, e.g., WO2014/145156, incorporated herein by reference.

As used herein, the term “eutectic system” refers to a mixture of chemical compounds or elements that has a single chemical composition that melts at a lower temperature than the other components of the mixture. A composition comprising a eutectic is known as the eutectic composition and its melting temperature is known as the eutectic temperature. Eutectic compositions often have higher stability and/or dissolution rates than their non-eutectic counterparts. Because eutectics enhance dissolution, they can be employed to increase permeability in solid dispersions and dispersion systems.

As used herein, the term “about” refers to a value or parameter that includes (and describes) embodiments that are directed to that value or parameter per se. For example, description referring to “about X” includes description of “X.” As used herein, the term “about” permits a variation of ±10% within the range of the significant digit.

Any suitable route of administration may be employed for providing the subject with the pharmaceutical compositions of the methods of this disclosure. For example, sublingual, buccal, oral, rectal, vaginal, parenteral, transdermal, intranasal, inhalational and the like may be employed as appropriate. The term parenteral as used herein includes subcutaneous, intracutaneous, intravenous, intramuscular, intra-articular, intrasynovial, intrasternal, intrathecal, intralesional and intracranial administration or other infusion techniques. Dosage forms useful in the methods of this disclosure may include tablets, such as scored tablets, coated tablets, or orally dissolving tablets; thin films, powders, caplets, capsules (e.g. hard gelatin capsules), troches, dragees, dispersions, suspensions, solutions, suppositories, patches and the like, including sustained release, extended release, slow release, modified release formulations well-known in the art. In some embodiments, the dosage form is a sublingual tablet, a sublingual film, a liquid, sublingual powder, or a sublingual spray solution. In some embodiments, the dosage form is a sublingual tablet. In some embodiments, the dosage form is a sublingual film. In some embodiments, the dosage form is a sublingual powder. In some embodiments, the dosage form is a sublingual spray solution. In some embodiments, the dosage form is a liquid.

In some embodiments, the pharmaceutical compositions of the disclosure are formulated for transmucosal absorption and in some embodiments, sublingual absorption including sublingual tablets, sublingual thin film formulations, sublingual powders, sublingual spray solutions. These embodiments bypass first-pass hepatic metabolism of cyclobenzaprine by cytochrome P-450 3A4 as a CYP3A substrate. In some embodiments, a controlled-release pharmaceutical composition of is used in the methods of this disclosure. That formulation is capable of releasing cyclobenzaprine into a subject at a desired rate, so as to maintain a substantially constant or desired pharmacological activity for a given period of time, to reduce or remove the effect of food on absorption, and/or to provide elimination of the drug and metabolites from the body with a reduced terminal elimination phase. As used herein, a “controlled-release component” is a compound such as a lipid or mixture of lipids, liposome and/or microsphere that induces the controlled-release of cyclobenzaprine into the subject upon exposure to a certain physiological compound or condition. For example, the controlled-release component can be biodegradable, activated by exposure to a certain pH or temperature, by exposure to an aqueous environment, or by exposure to enzymes. An example of a controlled-release component which is activated by exposure to a certain temperature is a sol-gel. In this embodiment, cyclobenzaprine is incorporated into a sol-gel matrix that is a solid at room temperature. This sol-gel matrix is implanted into a subject having a body temperature high enough to induce gel formation of the sol-gel matrix, thereby releasing the active ingredient into the subject.

As used herein, the term “alcohol use disorder (AUD)” or “alcoholism” refers to a pattern of alcohol use that involves problems controlling drinking, being preoccupied with alcohol, continuing to use alcohol even when it causes problems, having to drink to get the same effect (e.g., drinking alcohol more often and/or drinking greater quantities of alcohol), or having withdrawal symptoms when consumption is rapidly decreased or stopped. AUD can lead to health and safety risks.

As used herein, the term “alcohol intoxication” refers to increased blood alcohol concentration in the bloodstream. The higher the blood alcohol concentration leads to increased impairment. Alcohol concentration causes behavioral problems and mental change which may include inappropriate behavior, unstable moods, impaired judgment, slurred speech, impaired attention or memory (e.g., blackouts), and poor coordination. Very high blood alcohol levels can lead to coma or even death.

As used herein, the term “alcohol withdrawal” refers a period of experiencing symptoms after prolonged and heavy use of alcohol that is stopped or greatly reduced. Alcohol withdrawal can occur within several hours to four or five days after reducing or stopping prolonged and heavy use of alcohol. Signs and symptoms include sweating, rapid heartbeat, hand tremors, problems sleeping, nausea and vomiting, hallucinations, restlessness and agitation, anxiety, and occasionally seizures. Symptoms can be severe enough to impair the ability to function at work or in social situations.

### Alcohol Use Disorder

Alcohol use disorder (AUD) can be diagnosed by criteria outlined in the Diagnostic and Statistical Manual of Mental Disorders (DSM). Under DSM-5, anyone meeting any two of the 11 criteria during the same 12-month period receives a diagnosis of AUD. The severity of AUD ranges from mild (i.e., 2 to 3 symptoms), moderate (i.e., 4 to 5 symptoms), or severe (i.e., 6 or more symptoms). A health professional can help assess for AUD. Questions that are asking during the assessment include:

In the past year, have you:
1. Had times when you ended up drinking more, or longer than you intended?
2. More than once wanted to cut down or stop drinking, or tried to, but couldn't?
3. Spent a lot of time drinking? Or being sick or getting over the aftereffects?
4. Wanted a drink so badly you couldn't think of anything else? (Experienced craving—a strong need, or urge, to drink?)
5. Found that drinking—or being sick from drinking—often interfered with taking care of your home or family? Or caused job troubles? Or school problems?
6. Continued to drink even though it was causing trouble with your family or friends?
7. Given up or cut back on activities that were important or interesting to you, or gave you pleasure, in order to drink?
8. More than once gotten into situations while or after drinking that increased your chances of getting hurt (such as driving, swimming, using machinery, walking in a dangerous area, or having unsafe sex)?
9. Continued to drink even though it was making you feel depressed or anxious or adding to another health problem? Or after having had a memory blackout?
10. Had to drink much more than you once did to get the effect you want? Or found that your usual number of drinks had much less effect than before?
11. Found that when the effects of alcohol were wearing off, you had withdrawal symptoms, such as trouble sleeping, shakiness, irritability, anxiety, depression, restlessness, nausea, or sweating? Or sensed things that were not there?

Symptoms associated with alcohol use disorder include 1) being unable to limit the amount of alcohol consumed; 2) wanting to cut down on how much alcohol is consumed or making unsuccessful attempts to do so; 3) spending a lot of time drinking, getting alcohol or recovering from alcohol use; 4) feeling a strong craving or urge to drink alcohol; 5) failing to fulfill major obligations at work, school or home due to repeated alcohol use; 6) continuing to drink alcohol even though the consumption is causing physical, social or interpersonal problems; 7) giving up or reducing social and work activities and hobbies; 8) using alcohol in situations where it's not safe (e.g., when driving or swimming); 9) developing a tolerance to alcohol so consumption is continued or increased to feel its effect or experiencing a reduced effect from the same amount; 10) experiencing withdrawal symptoms (e.g., nausea, sweating, shaking, and problem sleeping) when consumption is stopped, or drinking to avoid these symptoms.

### Method for Treating

Some embodiments of this disclosure refer to a method for treating alcohol use disorder (AUD) and associated symptoms, comprising administering to a subject in need thereof, a pharmaceutical composition comprising a therapeutically effective amount of cyclobenzaprine or a pharmaceutically acceptable salt thereof and a pharmaceutically acceptable carrier.

In some embodiments, the pharmaceutical composition is administered one or more times daily. In some embodiments, the pharmaceutical composition is administered once daily. In some embodiments, the pharmaceutical composition is administered two times daily. In some embodiments, the pharmaceutical composition is administered three times daily.

In some embodiments, the pharmaceutical composition comprises between 0.1 mg and 30 mg of cyclobenzaprine or a pharmaceutically acceptable salt thereof. In some embodiments, the pharmaceutical composition comprises between 1 mg and 20 mg of cyclobenzaprine or a pharmaceutically acceptable salt thereof. In some embodiments, the pharmaceutical composition comprises between 1 mg and 10 mg of cyclobenzaprine or a pharmaceutically acceptable salt thereof. In some embodiments, the pharmaceutical composition comprises between 1 mg and 20 mg of cyclobenzaprine or a pharmaceutically acceptable salt thereof. In some embodiments, the pharmaceutical composition comprises between 2 mg and 6 mg of cyclobenzaprine or a pharmaceutically acceptable salt thereof. In some embodiments, the pharmaceutical composition comprises less than 10 mg of cyclobenzaprine or a pharmaceutically acceptable salt thereof. In some embodiments, the pharmaceutical composition comprises less than 5 mg of cyclobenzaprine or a pharmaceutically acceptable salt thereof. In some embodiments, the pharmaceutical composition comprises about 5.6 mg of cyclobenzaprine or a pharmaceutically acceptable salt thereof. In some embodiments, the pharmaceutical composition comprises about 2.8 mg of cyclobenzaprine or a pharmaceutically acceptable salt thereof. In some embodiments, the pharmaceutical composition comprises about 5.6 mg of cyclobenzaprine salt. In some embodiments, the pharmaceutical composition comprises about 2.8 mg of cyclobenzaprine salt. In some embodiments, the pharmaceutical composition comprises about 5.6 mg of cyclobenzaprine acid salt. In some embodiments, the pharmaceutical composition comprises about 2.8 mg of cyclobenzaprine acid salt. In some embodiments, the pharmaceutical composition comprises about 5.6 mg of cyclobenzaprine HCl. In some embodiments, the pharmaceutical composition comprises about 2.8 mg of cyclobenzaprine HCl.

In some embodiments, the pharmaceutical composition is administered as two dosage units, and wherein each dosage unit comprises 2.8 mg of cyclobenzaprine or a pharmaceutically acceptable salt thereof. In some embodiments, the pharmaceutical composition is administered as two dosage units, and wherein each dosage unit comprises 2.8 mg of cyclobenzaprine salt. In some embodiments, the pharmaceutical composition is administered as two dosage units, and wherein each dosage unit comprises 2.8 mg of cyclobenzaprine acid salt. In some embodiments, the pharmaceutical composition is administered as two dosage units, and wherein each dosage unit comprises 2.8 mg of cyclobenzaprine HCl. In some embodiments, the pharmaceutical composition is administered simultaneously as two dosage units, and wherein the combined amount in the two dosage units is 5.6 mg of cyclobenzaprine or a pharmaceutically acceptable salt thereof. In some embodiments, the pharmaceutical composition is administered simultaneously as two dosage units, and wherein the combined amount in the two dosage units is 5.6 mg of cyclobenzaprine salt. In some embodiments, the pharmaceutical composition is administered simultaneously as two dosage units, and wherein the combined amount in the two dosage units is 5.6 mg of cyclobenzaprine acid salt. In some embodiments, the pharmaceutical composition is administered simultaneously as two dosage units, and wherein the combined amount in the two dosage units is 5.6 mg of cyclobenzaprine HCl.

In some embodiments, the pharmaceutical composition is administered sublingually, buccally, orally, intravenously, intramuscularly, subcutaneously, inhalationally, intranasally, transdermally, parenterally, rectally, or vaginally. In some embodiments, the pharmaceutical composition is administered sublingually. In some embodiments, the pharmaceutical composition is administered buccally. In some embodiments, the pharmaceutical composition is administered orally. In some embodiments, the pharmaceutical composition is administered intravenously. In some embodiments, the pharmaceutical composition is administered intramuscularly. In some embodiments, the pharmaceutical composition is administered subcutaneously. In some embodiments, the pharmaceutical composition is administered inhalationally. In some embodiments, the pharmaceutical composition is administered intranasally. In some embodiments, the pharmaceutical composition is administered transdermally. In some embodiments, the pharmaceutical composition is administered parenterally. In some embodiments, the pharmaceutical composition is administered rectally. In some embodiments, the pharmaceutical composition is administered vaginally.

In some embodiments, the pharmaceutical composition is administered for transmucosal absorption.

In some embodiments, the pharmaceutically acceptable salt is a cyclobenzaprine acid salt. In some embodiments, the cyclobenzaprine acid salt is cyclobenzaprine HCl.

In some embodiments, the cyclobenzaprine or the pharmaceutically acceptable salt thereof in the pharmaceutical composition comprises at least in part a eutectic with mannitol. In some embodiments, the eutectic comprises cyclobenzaprine HCl and mannitol. In some embodiments, the eutectic comprises 75%±2% cyclobenzaprine HCl and 25%±2% mannitol. In some embodiments, the eutectic comprises 65%±2% cyclobenzaprine HCl and 35%±2% mannitol. In some embodiments, the eutectic comprises 75%±2% cyclobenzaprine HCl and 25%±2% f3-mannitol. In some embodiments, the eutectic comprises 65%±2% cyclobenzaprine HCl and 35%±2% 6-mannitol.

In some embodiments, the pharmaceutical composition is administered in combination with behavioral or environmental intervention. In some embodiments, the pharmaceutical composition is administered before, during, or after detoxification.

1. A method for treating alcohol use disorder (AUD) and associated symptoms, comprising administering to a subject in need thereof, a pharmaceutical composition comprising a therapeutically effective amount of cyclobenzaprine or a pharmaceutically acceptable salt thereof and a pharmaceutically acceptable carrier.

2. The method of claim 1, wherein the pharmaceutical composition is administered one or more times daily.

3. The method of claim 2, wherein the pharmaceutical composition comprises between 0.1 mg and 30 mg of cyclobenzaprine or a pharmaceutically acceptable salt thereof.

4. The method of claim 2, wherein the pharmaceutical composition comprises between 1 mg and 20 mg of cyclobenzaprine or a pharmaceutically acceptable salt thereof.

5. The method of claim 2, wherein the pharmaceutical composition comprises less than 10 mg of cyclobenzaprine or a pharmaceutically acceptable salt thereof.

6. The method of claim 2, wherein the pharmaceutical composition comprises less than 5 mg of cyclobenzaprine or a pharmaceutically acceptable salt thereof.

7. The method of claim 2, wherein the pharmaceutical composition comprises about 5.6 mg of cyclobenzaprine or a pharmaceutically acceptable salt thereof.

8. The method of claim 2, wherein the pharmaceutical composition comprises about 2.8 mg of cyclobenzaprine or a pharmaceutically acceptable salt thereof.

9. The method of claim 7, wherein the pharmaceutical composition is administered as two dosage units, and wherein each dosage unit comprises 2.8 mg of cyclobenzaprine or a pharmaceutically acceptable salt thereof.

10. The method of claim 7, wherein the pharmaceutical composition is administered simultaneously as two dosage units, and wherein the combined amount in the two dosage units is 5.6 mg of cyclobenzaprine or a pharmaceutically acceptable salt thereof.

11. The method of claim 1, wherein the pharmaceutical composition is administered once daily.

12. The method of claim 1, wherein the pharmaceutical composition is administered sublingually, buccally, orally, intravenously, intramuscularly, subcutaneously, inhalationally, intranasally, transdermally, parenterally, rectally, or vaginally.

13. The method of claim 11, wherein the pharmaceutical composition is administered for transmucosal absorption.

14. The method of claim 11, wherein the pharmaceutical composition is administered sublingually.

15. The method of claim 12, wherein the pharmaceutically acceptable salt comprises a cyclobenzaprine acid salt and the pharmaceutical composition further comprises a basifying agent.

16. The method of claim 15, wherein the cyclobenzaprine acid salt is cyclobenzaprine HCl.

17. The method of claim 1, wherein the cyclobenzaprine or the pharmaceutically acceptable salt thereof in the pharmaceutical composition comprises at least in part a eutectic of cyclobenzaprine or a pharmaceutically acceptable salt thereof and mannitol.

18. The method of claim 1, wherein the pharmaceutical composition is administered in combination with behavioral or environmental intervention.

19. The method of claim 1, wherein the pharmaceutical composition is administered before, during, or after detoxification.
